(19) United States
(12) Patent Application Publication
(10) Pub. No.: US 2023/0173088 A1
(43) Pub. Date: Jun. 8, 2023

(54) COMPOSITIONS AND METHODS FOR TREATING AND SUPPRESSING ALLERGIC RESPONSES

(71) Applicant: IgGenix, Inc., South San Francisco, CA (US)

(72) Inventor: Stephen R. Quake, Stanford, CA (US)

(21) Appl. No.: 18/073,663

(22) Filed: Dec. 2, 2022

Related U.S. Application Data
(60) Provisional application No. 63/287,344, filed on Dec. 8, 2021.

(51) Int. Cl. A61K 47/68

(52) U.S. Cl. CPC A61K 47/6843; A61K 47/6811; A61K 47/6803; A61K 47/6835

(57) ABSTRACT

The invention generally relates to therapeutic methods for treating and suppressing allergic responses. The invention provides therapeutic compositions and methods for treating and suppressing allergic responses using antibodies, antibody fragments thereof, or other products of the immune system coupled with a steric inhibitor to bind to an epitope on an allergen. Because of the steric inhibitor, the composition prevents one or more IgE antibodies from binding to the allergen. By blocking one or more IgE antibodies from binding to the allergen, the composition prevents IgE-mediated cross-linking and degranulation of mast cells and basophils, thus inhibiting anaphylaxis.

### FIELD OF INVENTION

The invention generally relates to therapeutic compositions for treating allergic responses.

### BACKGROUND

Allergies are characterized by hypersensitivity of the immune system to typically harmless substances in the environment. In general, an allergic reaction occurs when the immune system overreacts to the presence of a substance (an allergen) that, absent the allergy, would not cause a reaction. Food, insect bites, and medications may cause allergic reactions, with food allergies being a significant problem.

The United States Food and Drug Administration recognizes eight foods commonly implicated in allergy: peanuts, tree nuts, eggs, milk, shellfish, fish, wheat, and soy. In addition, there are also many significant non-food allergies, including, but not limited to, pollen (e.g., ragweed, trees, and grasses), animals (e.g., animal dander), molds, metals, and latex.

An allergic reaction can be caused by any form of contact with the allergen including ingestion, inhalation, or direct contact. Certain allergens may produce a systemic allergic response that may manifest as skin reactions, bronchoconstriction, swelling, low blood pressure, coma, and even death. Allergy to plant-derived foods is a highly complex disorder with clinical manifestations ranging from mild oral, gastrointestinal, and cutaneous symptoms to life-threatening systemic conditions. It is understood that allergic individuals must practice strict lifelong avoidance, thus impairing major life activities and quality of life. Allergies, especially food allergies, continue to be a threat to public health. Allergic diseases constitute a significant cause of morbidity worldwide and a considerable burden on health and medical systems.

### SUMMARY

The invention provides therapeutic compositions and methods for treating and suppressing allergic responses using antibodies, antibody fragments thereof, or other products of the immune system coupled with a steric inhibitor to bind to an epitope on an allergen. Compositions of the invention substantially prevent IgE antibodies from binding to the allergen. By blocking IgE antibodies compositions of the invention prevent IgE-mediated cross-linking and degranulation of mast cells and basophils, thus inhibiting allergic reactions, including anaphylaxis. Compositions and methods of the invention prevent or suppress an allergic response by both stoichiometrically competing with endogenous IgE antibodies or sterically blocking allergen binding to IgE which prevents cross-linking, degranulation, and anaphylaxis.

In one aspect, compositions of the invention involve antibodies linked to large molecular structures. For cross-linking and degranulation to occur, IgE receptors on mast cells must bind to two or more epitopes on an allergen. The invention provides therapeutic compositions that inhibit mast cells from binding to a second epitope. Compositions result in specific antigen binding, while at the same time blocking further IgE binding. The compositions may include a binding moiety linked to the antibody or antigen-binding fragment thereof, wherein the binding moiety binds to a macromolecule after in vivo delivery. The binding moiety portion of the composition binds to an endogenous in vivo molecular structure present in the body thus becoming a steric inhibitor coupled to the antibody or antigen-binding fragment, preventing IgE-mediated degranulation of mast cells and/or basophils. The binding moiety may bind to any macromolecule with a long chain, branched, multimeric, or quaternary molecular structure with sufficient size to inhibit further antigen binding to, and crosslinking of, IgE receptor complexes on mast cells and basophils. For example, the steric inhibitor may be, but is not limited to, a carbohydrate, protein, lipid, nucleic acid, oligonucleotide, long-chain lipid, or fatty acid. The binding moiety may be a ligand, such as an albumin-binding ligand. Alternatively, the ligand may be a human serum albumin which, once delivered, binds to fatty acids. Further, the ligand may be a lectin which, once delivered, binds to one or more sugars.

Compositions of the invention have sufficient size to inhibit binding and cross-linking of IgE receptor complexes on mast cells and basophils. The steric inhibitor portion of the composition may be from about 10 to about 1000 times larger than the antibody portion or antigen-binding fragment. Therefore, steric inhibition of a second epitope on the allergen from binding IgE is achieved through the large physical size of the composition.

The anaphylactic response arises when effector B cells of a person's immune system produce IgE immunoglobulin specific to an allergen. In IgE-mediated responses, exposure to the allergenic substance induces IgE production by the effector B cells of a person's immune system. The immune system hyperproduces IgE after initial contact with the allergen. An Fc portion of the IgE molecules binds to Fc receptors on mast cells in mucosal tissues lining body surfaces and cavities, as well as basophils in the circulation. The antigen-induced aggregation of IgE bound to the Fc receptors on mast cells and basophils is known as cross-linking. In other words, cross-linking occurs when multiple IgE/Fc complexes bind to the allergen. Cross-linking activates mast cells and basophils to undergo rapid degranulation which releases the inflammatory mediators within minutes of activation. Those mediators promote vasodilation, vascular permeability, and responses such as bronchoconstriction, which can lead to anaphylactic shock and death.

The allergic response can also result in a natural feedback loop in which basophils and mast cells degranulate in response to contact with IgE, releasing IL-4 which may promote switching of B cells and an increase in IgE. According to the invention, the coupling of the steric blocker with the antibody or antigen-binding fragment of an antibody prevents IgE/Fc complexes from becoming cross-linked which prevents mast cells and basophils from degranulating, thus inhibiting anaphylaxis.

In one aspect, the invention provides compositions for treating allergies comprising an antibody or antigen-binding fragment thereof that specifically binds an epitope on an allergen, and includes a steric inhibitor coupled to the antibody or antigen-binding fragment that prevents IgE-mediated cross-linking and degranulation of mast cells and basophils.

In certain embodiments, the antibody, or antigen-binding fragment thereof of the composition, is a monoclonal antibody, a polyclonal antibody, or a synthetic antibody such as a recombinant antibody, nucleic acid aptamer, or non-immunoglobulin antibody. Alternatively, the antibody or antigen-binding fragment thereof, is an immunoglobulin molecule selected from the group consisting of IgG, IgA, IgM, and a fragment thereof. Further, the IgG antibody is a monoclonal antibody that binds to Ara h 1, Ara h 2, Ara h 3, or Ara h 6.

The steric inhibitor coupled to the antibody or antigen-binding fragment thereof of the composition, is any molecule with sufficient size to inhibit further antigen binding to, and cross-linking of, IgE receptor complexes on mast cells and basophils. In certain embodiments, the steric inhibitor is a macromolecule or any long chain, branched, multimer, or quaternary molecular structure such as, but not limited to, a carbohydrate, polymer, or oligonucleotide. In other embodiments, the steric inhibitor is polyethylene glycol or albumin. The steric inhibitor can be covalently linked to a hinge region of the antibody or antigen-binding fragment thereof. In a further embodiment, the steric inhibitor of the composition is coupled to the antibody or antigen-binding fragment thereof via a linker.

Compositions and methods are designed to alleviate and/or prevent an allergic response. It is noted that compositions and methods described herein are useful to prevent and treat all forms of allergies and associated allergens. The specific allergen may include, but is not limited to, a food allergen, a plant allergen, a fungal allergen, an animal allergen, a dust mite allergen, a drug allergen, a cosmetic allergen, or a latex allergen. In some embodiments, the compositions specifically bind to a food allergen, such as a milk allergen, an egg allergen, a nut allergen, a fish allergen, a shellfish allergen, a soy allergen, a legume allergen, a seed allergen, or a wheat allergen, and sterically block further binding, cross-linking, and degranulation. In some embodiments, compositions specifically bind to a peanut allergen, and sterically block further binding, cross-linking, and degranulation. In other embodiments, the allergen is an environmental allergen. In some embodiments, the compositions are cross-reactive with more than one allergen.

In some embodiments, the composition is formulated for intranasal, transdermal, oral, or intravenous delivery. Compositions of the invention may also be encapsulated in a microparticle. The microparticle may be a lipid nanoparticle.

In another aspect, the invention provides a composition for treating allergies, the composition comprising a binding moiety linked to an antibody or antigen-binding fragment thereof that specifically binds an epitope on an allergen wherein when the antibody is delivered into a body, the binding moiety binds to a molecular structure present in the body thus becoming a steric inhibitor coupled to the antibody or antigen-binding fragment and preventing IgE-mediated degranulation of mast cells and/or basophils.

In certain aspects, the binding moiety binds to any macromolecule with a long chain, branched, multimeric, or quaternary molecular structure with sufficient size to inhibit further antigen binding to, and crosslinking of, IgE receptor complexes on mast cells and basophils. The binding moiety may be a ligand. Further, the ligand may be an albumin-binding ligand. Alternatively, the ligand may be a human serum albumin which, once delivered, binds to fatty acids. In embodiments, the ligand may be a lectin which, once delivered, binds to one or more sugars.

In another aspect, the invention provides methods for treating allergies, the methods comprising providing to a subject having an allergy or at risk of having an allergic reaction a composition comprising an antibody or antigen-binding fragment thereof that specifically binds an epitope on an allergen, and a steric inhibitor coupled to the antibody or antigen-binding fragment that prevents IgE-mediated degranulation of mast cells and/or basophils.

In certain embodiments, compositions of the invention comprise an antibody, or antigen-binding fragment thereof that is a monoclonal antibody, a polyclonal antibody, or a synthetic antibody such as a recombinant antibody, nucleic acid aptamer, or non-immunoglobulin antibody. Alternatively, the antibody or antigen-binding fragment thereof, is an immunoglobulin molecule selected from the group consisting of IgG, IgA, IgM, and a fragment thereof. Further, the IgG antibody is a monoclonal antibody that binds to Ara h 1, Ara h 2, Ara h 3, or Ara h 6.

Compositions of the invention includes a steric inhibitor coupled to the antibody or antigen-binding fragment thereof. The steric inhibitor is any molecule with sufficient size to inhibit further antigen binding to, and cross-linking of, IgE receptor complexes on mast cells and basophils. In some embodiments, the steric inhibitor is a macromolecule, or any long chain, branched, multimer, or quaternary molecular structure such as, but not limited to, a carbohydrate, polymer, or oligonucleotide. In other embodiments, the steric inhibitor coupled to the antibody comprises polyethylene glycol or albumin. The steric inhibitor may also be covalently linked to a hinge region of the antibody or antigen-binding fragment thereof. In a further embodiment, the steric inhibitor is coupled to the antibody or antigen-binding fragment via a linker.

Methods of the invention are useful to treat any allergy, including but not limited to food allergies, animal allergen, environmental allergies. In some embodiments, compositions of the invention specifically bind to a peanut allergen, and sterically block further binding, cross-linking, and degranulation. In some embodiments, the compositions are cross-reactive with more than one allergen. In all cases, the composition of method includes a steric inhibitor to sterically block IgE-mediated cross-linking and degranulation. As noted above, the methods described herein are useful to prevent and treat all forms of allergies and associated allergens.

Compositions of the invention may be formulated for intranasal, transdermal, oral, or intravenous delivery. Compositions of the invention are formulated to facilitate delivery. In a preferred embodiment, compositions of the invention are encapsulated in a microparticle, such as a lipid nanoparticle. Compositions of the invention comprise a pharmaceutically-acceptable adjuvant, diluent, or carrier. Methods of the invention comprise administering a therapeutically effective amount of the composition to an individual in need thereof.

In another aspect, the invention provides methods for treating allergies, the methods comprising providing to a subject having an allergy or at risk of having an allergic reaction a composition comprising a binding moiety linked to an antibody or antigen-binding fragment thereof, wherein the binding moiety binds to a macromolecule after in vivo delivery. The binding moiety portion of the composition binds to an endogenous in vivo molecular structure present in the body thus becoming a steric inhibitor coupled to the antibody or antigen-binding fragment, preventing IgE-mediated degranulation of mast cells and/or basophils.

In various embodiments, the binding moiety binds to any macromolecule with a long chain, branched, multimeric, or quaternary molecular structure with sufficient size to inhibit further antigen binding to, and crosslinking of, IgE receptor complexes on mast cells and basophils. The binding moiety may be a ligand. Further, the ligand may be an albumin-binding ligand. Alternatively, the ligand may be a human serum albumin which, once delivered, binds to fatty acids. In embodiments, the ligand may be a lectin which, once delivered, binds to one or more sugars.

In other embodiments, compositions of the invention comprise a functionalized antibody, or antigen-binding fragment thereof that is a monoclonal antibody, a polyclonal antibody, or a synthetic antibody, such as a recombinant antibody, nucleic acid aptamer, or non-immunoglobulin antibody. Alternatively, the antibody or antigen-binding fragment thereof, is an immunoglobulin molecule selected from the group consisting of IgG, IgA, IgM, and a fragment thereof. Further, the IgG antibody is a monoclonal antibody that binds to Ara h 1, Ara h 2, Ara h 3, or Ara h 6.

### DETAILED DESCRIPTION

The invention provides therapeutic compositions and methods for treating and suppressing allergic responses using antibodies, antibody fragments thereof, or other products of the immune system coupled with a steric inhibitor to bind to an epitope on an allergen. Compositions of the invention prevent IgE antibodies from binding to an allergen. By blocking IgE antibodies from binding to the allergen, the composition prevents IgE-mediated cross-linking and degranulation of mast cells and basophils, thus inhibiting anaphylaxis.

Mast cells are a type of white blood cell that is found in connective tissues throughout the body, particularly under the skin, near blood vessels and lymph vessels, in nerves, and in the lungs and intestines. Basophils are a type of bone marrow-derived circulating leukocyte. Mast cells and basophils contain granules, which are secretory vesicles found within the cells. These granules contain inflammatory mediators such as histamine, proteases, lipid mediators, and cytokines. Thus, mast cells and basophils play a pivotal role in an allergic response.

An allergic response is an inappropriate response of the immune system to a normally harmless substance referred to as an allergen. An allergen is a type of antigen which induces an immune response in the body, particularly the production of antibodies. In the immune system, antigen receptors on immune cells, such as effector B cells, bind specific epitopes of an antigen. An epitope is the part of the antigen that the immune system recognizes which elicits the immune response. The literature reports that common allergens may each have some number of different epitopes and that numerous laboratory assays are used for epitope discovery (see, e.g., Matsuo, 2015, Allergology Int 64(4):332-343, incorporated by reference).

Many allergic responses such as anaphylaxis, allergic rhinitis (hay fever), some food allergies, and allergic asthma involve IgE and T helper 2 (TH2) cells that recognize antigenic epitopes of allergens. In IgE-mediated responses, exposure to the allergenic substance induces IgE production by the effector B cells of a person's immune system. The immune system hyperproduces IgE after initial contact with the allergen. An Fc portion of the IgE molecules binds to Fc receptors on mast cells in mucosal tissues lining body surfaces and cavities, as well as basophils in the circulation. The antigen-induced aggregation of IgE bound to the Fc receptors on mast cells and basophils is known as cross-linking. In other words, cross-linking occurs when multiple IgE/Fc complexes bind to the allergen. Cross-linking activates mast cells and basophils to undergo rapid degranulation which releases the inflammatory mediators within minutes of activation. Those mediators promote vasodilation, vascular permeability, and responses such as bronchoconstriction, which can lead to anaphylactic shock and death.

The allergic response can also result in a natural feedback loop where, for example, basophils and mast cells degranulate in response to contact with IgE, they release IL-4 which may promote switching of B cells, leading to an increase in IgE.

In one aspect, the invention provides compositions comprising an antibody or antigen-binding fragment thereof that specifically binds an epitope on an allergen, and includes a steric inhibitor coupled to the antibody or antigen-binding fragment that prevents IgE-mediated cross-linking and degranulation of mast cells and basophils. Therefore, the compositions of the invention are configured to block allergen binding to IgE, outcompete endogenous IgE for allergen binding, and sterically inhibit further allergen binding.

The antibody, or antigen-binding fragment thereof, of the composition may be a monoclonal antibody, a polyclonal antibody, or a synthetic antibody such as a recombinant antibody, nucleic acid aptamer, or non-immunoglobulin antibody. Methods of making and purifying antibodies are known in the art and were developed by 1980s as described Harlow and Lane, 1988, Antibodies: A Laboratory Manual, CSHP, incorporated by reference.

As is known in the art, a monoclonal antibody is an antibody made by cloning a unique white blood cell, wherein all antibodies derived this way trace back to a unique parent cell. Monoclonal antibodies can have monovalent affinity, binding only to the same epitope. Monoclonal antibodies may be isolated or purified using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, Cold Spring Harbor Laboratory, incorporated by reference. See also Olsson, 1984, Human monoclonal antibodies: Methods of production and some aspects of their application in oncology, Med. Oncol. & Tumor Pharmacother 1, 235, incorporated by reference. These B cells are typically sourced from animals, usually mice. After cell fusion, large numbers of clones are screened and selected based on antigen specificity and immunoglobulin class.

Such hybridomas may be stored frozen and cultured as needed to produce the specific monoclonal antibody. As is described below, monoclonal antibodies may be deployed therapeutically in methods of the invention. Those immunoglobulins exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid. It should be noted that the methods of deriving nucleic acids, including a nucleic acid sequence, encoding the allergen-specific antibody, derived from sequences identified from isolated single B cells from a human subject who is allergic to the specific allergen are described in International PCT Application No. PCT/US2019/032951 (published as WO 2019/222679), the disclosure of which is incorporated by reference herein in its entirety. In particular, such methods include combining single cell RNA sequencing (scRNA-seq) with functional antibody assays to elucidate mechanisms underlying the regulation of IgE and to discover high affinity, cross-reactive allergen-specific antibodies.

Polyclonal antibodies are made using several different immune cells and have affinity for the same antigen but different epitopes. Polyclonal antibodies may be prepared and purified by methods known to one skilled in the art such as by injecting an antigen/adjuvant conjugate into an animal of choice to initiate an amplified immune response, extracting blood, and purifying for the antibody of interest. Many methodologies exist for polyclonal antibody production in laboratory animals and are known to a person skilled in the art, such as is described in Dunbar, 1990, Preparation of polyclonal antibodies. Methods Enzymol 182:663-70, incorporated by reference. See also Newcombe C, 2007, Antibody production: polyclonal-derived biotherapeutics. J Chromatogr B Analyt Technol Biomed Life Sci. March 15; 848(1):2-7, incorporated by reference.

Synthetic antibodies include recombinant antibodies, nucleic acid aptamers, and non-immunoglobulin protein scaffolds. Synthetic antibodies may be purchased commercially. Methods for making and purifying synthetic antibodies are known in the art and can be found, for example, in Takeuchi T, 2018, Beyond natural antibodies—a new generation of synthetic antibodies created by post-imprinting modification of molecularly imprinted polymer, s. Chem Commun (Camb) 54(49):6243-6251, incorporated by reference. For example, recombinant antibodies are monoclonal antibodies generated in vitro using synthetic genes. Recombinant monoclonal antibodies may be purchased, or prepared by recovering antibody genes from source cells, amplifying and cloning the genes into an appropriate high-yield expression vector, and introducing the vector into an expression host, such as bacteria, yeast, or mammalian cell lines generate adequate amounts of functional antibodies.

Non-immunoglobulin derived synthetic antibodies may be generated either from nucleic acids, as in the case of aptamers or from non-immunoglobulin protein scaffolds/peptide aptamers, into which hypervariable loops are inserted to form the antigen binding site. Constraining the hypervariable binding loop at both ends within the protein scaffold improves the binding affinity and specificity of the synthetic antibody to levels comparable to or exceeding that of a natural antibody. Common advantages of these molecules compared to use of the typical antibody structure include a smaller size, giving improved tissue penetration, rapid generation times of weeks compared to months for natural and recombinant antibodies and cheaper costs.

Importantly, the compositions of the invention provide therapeutic benefits by binding inhibitory receptors on mast cells and/or basophils and sterically inhibiting further allergen binding to, and cross-linking of, IgE receptor complexes on mast cells and basophils, thus preventing degranulation and anaphylaxis. Compositions of the invention therefore include a steric inhibitor coupled to the antibody or antigen-binding fragment thereof.

Steric inhibition of antigen binding is achieved through the large physical size of the composition as compared to the allergen. Steric hinderance is the slowing or prevention of a binding reaction due to steric bulk, usually manifested in intramolecular interactions. Compositions of the invention utilize steric inhibition by coupling a steric inhibitor to the antibody or antigen-binding fragment thereof to prevent antibody binding to their targets.

Compositions of the invention include a steric inhibitor coupled to the antibody or antigen-binding fragment thereof. The steric inhibitor may be any macromolecule with a long chain, branched, multimeric, or quaternary molecular structure with sufficient size to inhibit further antigen binding to, and crosslinking of, IgE receptor complexes on mast cells and basophils. For example, the steric inhibitor may be, but is not limited a carbohydrate, polymer, protein, lipid, nucleic acid, oligonucleotide, long-chain lipid, fatty acid, or a biocompatible synthetic polymer. For example, the steric inhibitor may be any number of times larger than the coupled antibody or antigen-binding fragment thereof, as long as it is of a size sufficient to provide steric hinderance of antigen binding to receptor complexes to prevent cross-linking and degranulation. The steric inhibitor may be covalently attached to a region of the antibody or antigen-binding fragment thereof sufficient to provide the physical bulk and orientation suitable for steric hinderance.

The biochemistry of attaching the steric inhibitor to the antibody or antigen-binding fragment thereof is known to the person skilled in the art. For example, in one embodiment, the steric inhibitor coupled to the antibody or antigen-binding fragment thereof may comprise a polymer such as a polymer of polyethylene glycol (PEG). The PEG polymer may be bis-maleimide PEG (BM-PEG). BM-PEG is a thiol reactive homobifunctional-containing two identical functional groups at both ends-PEG derivative selective for thiol groups on cysteine side chains. PEG (Maleimide)2 undergoes thiol PEGylation reactions with thiol-containing molecules at pH 5.0-6.5. Homobifunctional PEG derivatives have numerous applications as crosslinkers for PEGylation of proteins and peptides, nanoparticle and surface modifications. As is known to persons skilled in the art, PEGylation is the process of covalently binding PEG moieties to molecules, most typically peptides proteins, antibodies, and antibody fragments, such as described in Harris J M, 2001, Pegylation: a novel process for modifying pharmacokinetics, Clin Pharmacokinet 40(7):539-51, incorporated by reference. PEGylation produces alterations in the physiochemical properties including conformation, size, and molecular weight. PEGylation can influence the binding affinity of the therapeutic moiety to the cell receptors and can alter the absorption and distribution patterns. The steric inhibitor may also be covalently linked to a hinge or other region of the antibody or fragment thereof.

In other embodiments, the steric inhibitor may comprise albumin. Albumin is a family of globular proteins, the most common of which are the serum albumins. All the proteins of the albumin family are water-soluble, moderately soluble in concentrated salt solutions, and experience heat denaturation. Albumin is composed of a single polypeptide chain, folded so as to form three or four spherical units. The albumin-binding domain is a small, three-helical protein domain found in various surface proteins expressed by gram-positive bacteria. Albumin can also form ionic bonds. Non-covalent bonding by albumin is important and several homologous domains have been identified. See Nilvebrant, J., 2013, The albumin-binding domain as a scaffold for protein engineering, Computational and structural biotechnology journal 6, e201303009, incorporated by reference herein.

In still other embodiments, the steric inhibitor is coupled to the antibody, or antigen-binding fragment thereof, by a linker. Linkers or spacers are short amino acid sequences employed to form stable covalently linked dimers, and to connect two independent domains that create a ligand-binding site or recognition sequence. As is known to a person skilled in the art, recombinant DNA technology makes it possible to fuse two interacting partners with the introduction of artificial linkers. For example, as noted in Reddy Chichili, 2013, Linkers in the structural biology of protein-protein interactions, Protein science: a publication of the Protein Society 22(2), 153-167, incorporated by reference herein, Gly-rich linkers are flexible, connecting various domains in a single protein without interfering with the function of each domain. Gly-rich linkers can create a covalent link between the proteins to form a stable protein-protein complex. Gly-rich linkers are also employed to form stable covalently linked dimers, and to connect two independent domains that create a ligand-binding site or recognition sequence.

Because an allergic reaction involves mast cells or basophils presenting IgE-bound Fc receptors that bind to and are cross-linked by IgE, in embodiments, compositions may include an antibody fragment, such as an IgE fragment, that may bind an allergen but that does not bind and cross-link Fc receptors. Antibody fragments are proteins that form part of the antigen recognition site. Antibody fragments may be produced in genetically modified bacteriophages, bacteria, fungi, or plants, such as is described in Joosten, V., 2003, The production of antibody fragments and antibody fusion proteins by yeasts and filamentous fungi, Microb Cell Fact 2, 1, incorporated by reference.

In other embodiments, the antibody or antigen-binding fragment thereof, is an immunoglobulin molecule selected from the group consisting of IgG, IgA, IgM, and a fragment thereof. Immunoglobulin G (IgG) is the most common type of antibody found in blood circulation. As noted above, IgG antibodies are created and released by plasma B cells. Each IgG antibody has two paratopes, or antigen-binding sites. Immunoglobulin A (IgA) is an antibody that plays a crucial role in the immune function of mucous membranes. The amount of IgA produced in association with mucosal membranes is greater than all other types of antibody combined. Immunoglobulin M (IgM) is the largest antibody, and it is the first antibody to appear in the response to initial exposure to an antigen. The IgG, IgA, IgM antibodies or fragment thereof, may be produced by any of the methods described above.

In some embodiments, the IgG antibody is a monoclonal antibody that binds to Ara h 1, Ara h 2, Ara h 3, or Ara h 6. Peanut causes one of the most serious food allergies. Ara h 1, Ara h 2, Ara h 3, and Ara h 6 belong to the peanut seed storage protein classes conarachin, conglutin and arachin. Ara h 1, Ara h 2, and Ara h 3 are classified as the major peanut allergens which can be recognized by more than 50% of peanut-allergic patients. See Burks W, Sampson H A, 1998, Peanut Allergens, Allergy 53:725-730, incorporated by reference herein. See also Rabjohn P, et al., 1999, Molecular cloning and epitope analysis of the peanut allergen Ara h 3, J Clin Invest 103:535-542, incorporated by reference herein; see also Barre A, et al., 2005, Molecular modelling of the major peanut allergen Ara h 1 and other homotrimeric allergens of the cupin superfamily: a structural basis for their IgE-binding cross-reactivity, Biochimie 87:499-506, incorporated by reference herein. Ara h 3 is recognized by serum IgE from approximately 44-54% of different patient populations with a history of peanut sensitivity. See Ratnaparkhe, M. B., et. al., 2014, Comparative and evolutionary analysis of major peanut allergen gene families, Genome biology and evolution 6(9), 2468-2488, incorporated by reference herein.

The compositions are designed to alleviate and prevent an allergic response associated with specific allergens. In embodiments the allergen targeted comprises a food allergen. For example, the food allergen may be, but is not limited to, a milk allergen, an egg allergen, a nut allergen, a fish allergen, a shellfish allergen, a soy allergen, a legume allergen, a seed allergen, or a wheat allergen. In other embodiments, the allergen is pet allergen. In still other embodiments, the allergen is an environmental allergen such as, but not limited to, a plant allergen, a fungal allergen, a dust mite allergen, a drug allergen, a cosmetic allergen, or a latex allergen.

The composition may be formulated for intranasal, transdermal, oral, or intravenous delivery. Further, the composition may be encapsulated in a microparticle, such as a lipid nanoparticle. As described in Lengyel, 2019, Microparticles, Microspheres, and Microcapsules for advanced drug delivery, Sci. Pharm 87, 20, incorporated by reference herein, and known to the skilled artisan, microparticles, microspheres, and microcapsules are widely used constituents of multiparticulate drug delivery systems. Microparticles are generally in the 1-1000 μm size range, serve as multiunit drug delivery systems with well-defined physiological and pharmacokinetic benefits in order to improve the effectiveness, tolerability, and patient compliance.

In another aspect, the invention provides a composition in which the antibody, or antigen-binding fragment thereof, includes a binding moiety linked to the antibody or antigen-binding fragment thereof, and binds to a macromolecule within the patient after delivery into the patient. Specifically, the composition comprises a binding moiety linked to an antibody or antigen-binding fragment thereof, wherein the binding moiety binds to a macromolecule after in vivo delivery. The binding moiety portion of the composition binds to an endogenous in vivo molecular structure present in the body thus becoming a steric inhibitor coupled to the antibody or antigen-binding fragment, preventing IgE-mediated degranulation of mast cells and/or basophils. The binding moiety may bind to any macromolecule with a long chain, branched, multimeric, or quaternary molecular structure with sufficient size to inhibit further antigen binding to, and crosslinking of, IgE receptor complexes on mast cells and basophils. For example, the steric inhibitor may be, but is not limited a carbohydrate, protein, lipid, nucleic acid, oligonucleotide, long-chain lipid, or fatty acid.

In embodiments, the binding moiety linked to the antibody, or antigen-binding fragment thereof, is a ligand. The ligand may be an albumin-binding ligand such as described in Zorzi, A., 2017, Acylated heptapeptide binds albumin with high affinity and application as tag furnishes long-acting peptides, Nature Communications 8:16092, incorporated by reference herein. The ligand further may be a human serum albumin (HAS) which, once delivered, binds to fatty acids such as described in Fasano M, 2005, The extraordinary ligand binding properties of human serum albumin, IUBMB Life 57(12):787-96, incorporated by reference herein. Further, the ligand may be a lectin and, once delivered, binds to one or more sugars, as described in Raposo, D., 2021, Human lectins, their carbohydrate affinities and where to find them, Biomolecules 11, 188, incorporated by reference herein.

The antibody, or antigen-binding fragment thereof, of the composition may be a monoclonal antibody, a polyclonal antibody, or a synthetic antibody such as a recombinant antibody, nucleic acid aptamer, or non-immunoglobulin antibody. Methods of making and purifying antibodies are known in the art and were developed by 1980s as described Harlow and Lane, 1988, Antibodies: A Laboratory Manual, CSHP, incorporated by reference.

For example, a monoclonal antibody is an antibody made by cloning a unique white blood cell, wherein all antibodies derived this way trace back to a unique parent cell. Monoclonal antibodies can have monovalent affinity, binding only to the same epitope. Monoclonal antibodies may be isolated or purified using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, Cold Spring Harbor Laboratory, incorporated by reference. See also Olsson, 1984, Human monoclonal antibodies: Methods of production and some aspects of their application in oncology, Med. Oncol. & Tumor Pharmacother 1, 235, incorporated by reference. These B cells are typically sourced from animals, usually mice. After cell fusion, large numbers of clones are screened and selected on the basis of antigen specificity and immunoglobulin class.

Such hybridomas may be stored frozen and cultured as needed to produce the specific monoclonal antibody. As is described below, monoclonal antibodies may be deployed therapeutically in methods of the invention. Those immunoglobulins exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid. It should be noted that the methods of deriving nucleic acids, including a nucleic acid sequence, encoding the allergen-specific antibody, derived from sequences identified from isolated single B cells from a human subject who is allergic to the specific allergen are described in International PCT Application No. PCT/US2019/032951 (published as WO 2019/222679), the disclosure of which is incorporated by reference herein in its entirety. In particular, such methods include combining single cell RNA sequencing (scRNA-seq) with functional antibody assays to elucidate mechanisms underlying the regulation of IgE and to discover high affinity, cross-reactive allergen-specific antibodies.

Polyclonal antibodies are made using several different immune cells and have affinity for the same antigen but different epitopes. Polyclonal antibodies may be prepared and purified by methods known to one skilled in the art such as by injecting an antigen/adjuvant conjugate into an animal of choice to initiate an amplified immune response, extracting blood, and purifying for the antibody of interest. Many methodologies exist for polyclonal antibody production in laboratory animals and are known to a person skilled in the art, such as is described in Dunbar, 1990, Preparation of polyclonal antibodies. Methods Enzymol 182:663-70, incorporated by reference. See also Newcombe C, 2007, Antibody production: polyclonal-derived biotherapeutics. J Chromatogr B Analyt Technol Biomed Life Sci. March 15; 848(1):2-7, incorporated by reference.

Synthetic antibodies include recombinant antibodies, nucleic acid aptamers, and non-immunoglobulin protein scaffolds. Synthetic antibodies may be purchased commercially. Methods for making and purifying synthetic antibodies are known in the art and can be found, for example, in Takeuchi T, 2018, Beyond natural antibodies—a new generation of synthetic antibodies created by post-imprinting modification of molecularly imprinted polymer, s. Chem Commun (Camb) 54(49):6243-6251, incorporated by reference. For example, recombinant antibodies are monoclonal antibodies generated in vitro using synthetic genes. Recombinant monoclonal antibodies may be purchased, or prepared by recovering antibody genes from source cells, amplifying and cloning the genes into an appropriate high-yield expression vector, and introducing the vector into an expression host, such as bacteria, yeast, or mammalian cell lines generate adequate amounts of functional antibodies.

Non-immunoglobulin derived synthetic antibodies may be generated either from nucleic acids, as in the case of aptamers or from non-immunoglobulin protein scaffolds/peptide aptamers, into which hypervariable loops are inserted to form the antigen binding site. Constraining the hypervariable binding loop at both ends within the protein scaffold improves the binding affinity and specificity of the synthetic antibody to levels comparable to or exceeding that of a natural antibody. Common advantages of these molecules compared to use of the typical antibody structure include a smaller size, giving improved tissue penetration, rapid generation times of weeks compared to months for natural and recombinant antibodies and cheaper costs.

Because an allergic reaction involves mast cells or basophils presenting IgE-bound Fc receptors that bind to and are cross-linked by IgE, in embodiments, compositions may include an antibody fragment, such as an IgE fragment, that may bind an allergen but that does not bind and cross-link Fc receptors. Antibody fragments are proteins that form part of the antigen recognition site. Antibody fragments may be produced in genetically modified bacteriophages, bacteria, fungi, or plants, such as is described in Joosten, V., 2003, The production of antibody fragments and antibody fusion proteins by yeasts and filamentous fungi, Microb Cell Fact 2, 1, incorporated by reference.

In other embodiments, the antibody or antigen-binding fragment thereof, is an immunoglobulin molecule selected from the group consisting of IgG, IgA, IgM, and a fragment thereof. Immunoglobulin G (IgG) is the most common type of antibody found in blood circulation. As noted above, IgG antibodies are created and released by plasma B cells. Each IgG antibody has two paratopes, or antigen-binding sites. Immunoglobulin A (IgA) is an antibody that plays a crucial role in the immune function of mucous membranes. The amount of IgA produced in association with mucosal membranes is greater than all other types of antibody combined. Immunoglobulin M (IgM) is the largest antibody, and it is the first antibody to appear in the response to initial exposure to an antigen. The IgG, IgA, IgM antibodies or fragment thereof, may be produced by any of the methods described above.

In some embodiments, the IgG antibody is a monoclonal antibody that binds to Ara h 1, Ara h 2, Ara h 3, or Ara h 6. Peanut causes one of the most serious food allergies. Ara h 1, Ara h 2, Ara h 3, and Ara h 6 belong to the peanut seed storage protein classes conarachin, conglutin and arachin. Ara h 1, Ara h 2, and Ara h 3 are classified as the major peanut allergens which can be recognized by more than 50% of peanut-allergic patients. See Burks W, Sampson H A, 1998, Peanut Allergens, Allergy 53:725-730, incorporated by reference herein. See also Rabjohn P, et al., 1999, Molecular cloning and epitope analysis of the peanut allergen Ara h 3, J Clin Invest 103:535-542, incorporated by reference herein; see also Barre A, et al., 2005, Molecular modelling of the major peanut allergen Ara h 1 and other homotrimeric allergens of the cupin superfamily: a structural basis for their IgE-binding cross-reactivity, Biochimie 87:499-506, incorporated by reference herein. Ara h 3 is recognized by serum IgE from approximately 44-54% of different patient populations with a history of peanut sensitivity. See Ratnaparkhe, M. B., et. al., 2014, Comparative and evolutionary analysis of major peanut allergen gene families, Genome biology and evolution 6(9), 2468-2488, incorporated by reference herein.

The compositions are designed to alleviate and prevent an allergic response associated with specific allergens. In embodiments the allergen targeted comprises a food allergen. For example, the food allergen may be, but is not limited to, a milk allergen, an egg allergen, a nut allergen, a fish allergen, a shellfish allergen, a soy allergen, a legume allergen, a seed allergen, or a wheat allergen. In other embodiments, the allergen is pet allergen. In still other embodiments, the allergen is an environmental allergen such as, but not limited to, a plant allergen, a fungal allergen, a dust mite allergen, a drug allergen, a cosmetic allergen, or a latex allergen.

The composition may be formulated for intranasal, transdermal, oral, or intravenous delivery. Further, the composition may be encapsulated in a microparticle, such as a lipid nanoparticle. As described in Lengyel, 2019, Microparticles, Microspheres, and Microcapsules for advanced drug delivery, Sci. Pharm 87, 20, incorporated by reference herein, and known to the skilled artisan, microparticles, microspheres, and microcapsules are widely used constituents of multiparticulate drug delivery systems. Microparticles are generally in the 1-1000 μm size range, serve as multiunit drug delivery systems with well-defined physiological and pharmacokinetic benefits in order to improve the effectiveness, tolerability, and patient compliance.

In still another aspect, the invention provides a method for treating allergies, the method comprising providing to a subject having an allergy or at risk of having an allergic reaction a composition comprising an antibody or antigen-binding fragment thereof that specifically binds an epitope on an allergen, and a steric inhibitor coupled to said antibody or antigen-binding fragment that prevents IgE-mediated degranulation of mast cells and/or basophils.

As discussed above, the composition of the method includes an antibody or antigen-binding fragment thereof. The antibody, or antigen-binding fragment thereof, of the composition may be a monoclonal antibody, a polyclonal antibody, or a synthetic antibody such as a recombinant antibody, nucleic acid aptamer, or non-immunoglobulin antibody. Methods of making and purifying antibodies are known in the art and were developed by 1980s as described Harlow and Lane, 1988, Antibodies: A Laboratory Manual, CSHP, incorporated by reference.

For example, a monoclonal antibody is an antibody made by cloning a unique white blood cell, wherein all antibodies derived this way trace back to a unique parent cell. Monoclonal antibodies can have monovalent affinity, binding only to the same epitope. Monoclonal antibodies may be isolated or purified using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, Cold Spring Harbor Laboratory, incorporated by reference. See also Olsson, 1984, Human monoclonal antibodies: Methods of production and some aspects of their application in oncology, Med. Oncol. & Tumor Pharmacother 1, 235, incorporated by reference. These B cells are typically sourced from animals, usually mice. After cell fusion, large numbers of clones are screened and selected on the basis of antigen specificity and immunoglobulin class.

Such hybridomas may be stored frozen and cultured as needed to produce the specific monoclonal antibody. As is described below, monoclonal antibodies may be deployed therapeutically in methods of the invention. Those immunoglobulins exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid. It should be noted that the methods of deriving nucleic acids, including a nucleic acid sequence, encoding the allergen-specific antibody, derived from sequences identified from isolated single B cells from a human subject who is allergic to the specific allergen are described in International PCT Application No. PCT/US2019/032951 (published as WO 2019/222679), the disclosure of which is incorporated by reference herein in its entirety. In particular, such methods include combining single cell RNA sequencing (scRNA-seq) with functional antibody assays to elucidate mechanisms underlying the regulation of IgE and to discover high affinity, cross-reactive allergen-specific antibodies.

Polyclonal antibodies are made using several different immune cells and have affinity for the same antigen but different epitopes. Polyclonal antibodies may be prepared and purified by methods known to one skilled in the art such as by injecting an antigen/adjuvant conjugate into an animal of choice to initiate an amplified immune response, extracting blood, and purifying for the antibody of interest. Many methodologies exist for polyclonal antibody production in laboratory animals and are known to a person skilled in the art, such as is described in Dunbar, 1990, Preparation of polyclonal antibodies. Methods Enzymol 182:663-70, incorporated by reference. See also Newcombe C, 2007, Antibody production: polyclonal-derived biotherapeutics. J Chromatogr B Analyt Technol Biomed Life Sci. March 15; 848(1):2-7, incorporated by reference.

Synthetic antibodies include recombinant antibodies, nucleic acid aptamers, and non-immunoglobulin protein scaffolds. Synthetic antibodies may be purchased commercially. Methods for making and purifying synthetic antibodies are known in the art and can be found, for example, in Takeuchi T, 2018, Beyond natural antibodies—a new generation of synthetic antibodies created by post-imprinting modification of molecularly imprinted polymer, s. Chem Commun (Camb) 54(49):6243-6251, incorporated by reference. For example, recombinant antibodies are monoclonal antibodies generated in vitro using synthetic genes. Recombinant monoclonal antibodies may be purchased, or prepared by recovering antibody genes from source cells, amplifying and cloning the genes into an appropriate high-yield expression vector, and introducing the vector into an expression host, such as bacteria, yeast, or mammalian cell lines generate adequate amounts of functional antibodies.

Non-immunoglobulin derived synthetic antibodies may be generated either from nucleic acids, as in the case of aptamers or from non-immunoglobulin protein scaffolds/peptide aptamers, into which hypervariable loops are inserted to form the antigen binding site. Constraining the hypervariable binding loop at both ends within the protein scaffold improves the binding affinity and specificity of the synthetic antibody to levels comparable to or exceeding that of a natural antibody. Common advantages of these molecules compared to use of the typical antibody structure include a smaller size, giving improved tissue penetration, rapid generation times of weeks compared to months for natural and recombinant antibodies and cheaper costs.

Importantly, the compositions of the method provide therapeutic benefits by binding inhibitory receptors on mast cells and/or basophils and sterically inhibiting further allergen binding to, and cross-linking of, IgE receptor complexes on mast cells and basophils, thus preventing degranulation and anaphylaxis. Compositions of the method therefore include a steric inhibitor coupled to the antibody or antigen-binding fragment thereof.

Steric inhibition of antigen binding is achieved through the large physical size of the composition as compared to the allergen. Steric hinderance is the slowing or prevention of a binding reaction due to steric bulk, usually manifested in intramolecular interactions. Compositions of the invention utilize steric inhibition by coupling a steric inhibitor to the antibody or antigen-binding fragment thereof to prevent antibody binding to their targets.

Compositions of the method include a steric inhibitor coupled to the antibody or antigen-binding fragment thereof. The steric inhibitor may be any macromolecule with a long chain, branched, multimeric, or quaternary molecular structure with sufficient size to inhibit further antigen binding to, and crosslinking of, IgE receptor complexes on mast cells and basophils. For example, the steric inhibitor may be, but is not limited a carbohydrate, polymer, protein, lipid, nucleic acid, oligonucleotide, long-chain lipid, fatty acid, or a biocompatible synthetic polymer. For example, the steric inhibitor may be any number of times larger than the coupled antibody or antigen-binding fragment thereof, as long as it is of a size sufficient to provide steric hinderance of antigen binding to receptor complexes to prevent cross-linking and degranulation. The steric inhibitor may be covalently attached to a region of the antibody or antigen-binding fragment thereof sufficient to provide the physical bulk and orientation suitable for steric hinderance.

The biochemistry of attaching the steric inhibitor to the antibody or antigen-binding fragment thereof is known to the person skilled in the art. For example, in one embodiment, the steric inhibitor coupled to the antibody or antigen-binding fragment thereof may comprise a polymer such as a polymer of polyethylene glycol (PEG). The PEG polymer may be bis-maleimide PEG (BM-PEG). BM-PEG is a thiol reactive homobifunctional-containing two identical functional groups at both ends-PEG derivative selective for thiol groups on cysteine side chains. PEG (Maleimide)2 undergoes thiol PEGylation reactions with thiol-containing molecules at pH 5.0-6.5. Homobifunctional PEG derivatives have numerous applications as crosslinkers for PEGylation of proteins and peptides, nanoparticle, and surface modifications. As is known to persons skilled in the art, PEGylation is the process of covalently binding PEG moieties to molecules, most typically peptides proteins, antibodies, and antibody fragments, such as described in Harris J M, 2001, Pegylation: a novel process for modifying pharmacokinetics, Clin Pharmacokinet 40(7):539-51, incorporated by reference. PEGylation produces alterations in the physiochemical properties including conformation, size, and molecular weight. PEGylation can influence the binding affinity of the therapeutic moiety to the cell receptors and can alter the absorption and distribution patterns. The steric inhibitor may also be covalently linked to a hinge or other region of the antibody or fragment thereof.

In other embodiments, the steric inhibitor may comprise albumin. Albumin is a family of globular proteins, the most common of which are the serum albumins. All the proteins of the albumin family are water-soluble, moderately soluble in concentrated salt solutions, and experience heat denaturation. Albumin is composed of a single polypeptide chain, folded so as to form three or four spherical units. The albumin-binding domain is a small, three-helical protein domain found in various surface proteins expressed by gram-positive bacteria. Albumin can also form ionic bonds. Non-covalent bonding by albumin is important and several homologous domains have been identified. See Nilvebrant, J., 2013, The albumin-binding domain as a scaffold for protein engineering, Computational and structural biotechnology journal 6, e201303009, incorporated by reference herein.

In still other embodiments, the steric inhibitor is coupled to the antibody, or antigen-binding fragment thereof, by a linker. Linkers or spacers are short amino acid sequences employed to form stable covalently linked dimers, and to connect two independent domains that create a ligand-binding site or recognition sequence. As is known to a person skilled in the art, recombinant DNA technology makes it possible to fuse two interacting partners with the introduction of artificial linkers. For example, as noted in Reddy Chichili, 2013, Linkers in the structural biology of protein-protein interactions, Protein science: a publication of the Protein Society 22(2), 153-167, incorporated by reference herein, Gly-rich linkers are flexible, connecting various domains in a single protein without interfering with the function of each domain. Gly-rich linkers can create a covalent link between the proteins to form a stable protein-protein complex. Gly-rich linkers are also employed to form stable covalently linked dimers, and to connect two independent domains that create a ligand-binding site or recognition sequence.

Because an allergic reaction involves mast cells or basophils presenting IgE-bound Fc receptors that bind to and are cross-linked by IgE, in embodiments, compositions may include an antibody fragment, such as an IgE fragment, that may bind an allergen but that does not bind and cross-link Fc receptors. Antibody fragments are proteins that form part of the antigen recognition site. Antibody fragments may be produced in genetically modified bacteriophages, bacteria, fungi, or plants, such as is described in Joosten, V., 2003, The production of antibody fragments and antibody fusion proteins by yeasts and filamentous fungi, Microb Cell Fact 2, 1, incorporated by reference.

In other embodiments, the antibody or antigen-binding fragment thereof, is an immunoglobulin molecule selected from the group consisting of IgG, IgA, IgM, and a fragment thereof. Immunoglobulin G (IgG) is the most common type of antibody found in blood circulation. As noted above, IgG antibodies are created and released by plasma B cells. Each IgG antibody has two paratopes, or antigen-binding sites. Immunoglobulin A (IgA) is an antibody that plays a crucial role in the immune function of mucous membranes. The amount of IgA produced in association with mucosal membranes is greater than all other types of antibody combined. Immunoglobulin M (IgM) is the largest antibody, and it is the first antibody to appear in the response to initial exposure to an antigen. The IgG, IgA, IgM antibodies or fragment thereof, may be produced by any of the methods described above.

In some embodiments, the IgG antibody is a monoclonal antibody that binds to Ara h 1, Ara h 2, Ara h 3, or Ara h 6. Peanut causes one of the most serious food allergies. Ara h 1, Ara h 2, Ara h 3, and Ara h 6 belong to the peanut seed storage protein classes conarachin, conglutin and arachin. Ara h 1, Ara h 2, and Ara h 3 are classified as the major peanut allergens which can be recognized by more than 50% of peanut-allergic patients. See Burks W, Sampson H A, 1998, Peanut Allergens, Allergy 53:725-730, incorporated by reference herein. See also Rabjohn P, et al., 1999, Molecular cloning and epitope analysis of the peanut allergen Ara h 3, J Clin Invest 103:535-542, incorporated by reference herein; see also Barre A, et al., 2005, Molecular modelling of the major peanut allergen Ara h 1 and other homotrimeric allergens of the cupin superfamily: a structural basis for their IgE-binding cross-reactivity, Biochimie 87:499-506, incorporated by reference herein. Ara h 3 is recognized by serum IgE from approximately 44-54% of different patient populations with a history of peanut sensitivity. See Ratnaparkhe, M. B., et. al., 2014, Comparative and evolutionary analysis of major peanut allergen gene families, Genome biology and evolution 6(9), 2468-2488, incorporated by reference herein.

The compositions of the method are designed to alleviate and prevent an allergic response associated with specific allergens. In embodiments the allergen targeted comprises a food allergen. For example, the food allergen may be, but is not limited to, a milk allergen, an egg allergen, a nut allergen, a fish allergen, a shellfish allergen, a soy allergen, a legume allergen, a seed allergen, or a wheat allergen. In other embodiments, the allergen is pet allergen. In still other embodiments, the allergen is an environmental allergen such as, but not limited to, a plant allergen, a fungal allergen, a dust mite allergen, a drug allergen, a cosmetic allergen, or a latex allergen.

The composition of the method may be formulated for intranasal, transdermal, oral, or intravenous delivery. Further, the composition may be encapsulated in a microparticle, such as a lipid nanoparticle. As described in Lengyel, 2019, Microparticles, Microspheres, and Microcapsules for advanced drug delivery, Sci. Pharm 87, 20, incorporated by reference herein, and known to the skilled artisan, microparticles, microspheres, and microcapsules are widely used constituents of multiparticulate drug delivery systems. Microparticles are generally in the 1-1000 μm size range, serve as multiunit drug delivery systems with well-defined physiological and pharmacokinetic benefits in order to improve the effectiveness, tolerability, and patient compliance.

Methods of the invention provide for the administration of a therapeutically effective amount of a pharmaceutical formulation to a subject for preventing or treating an allergic response in the subject by providing the subject a dose of the composition. The formulation generally includes a composition comprising the composition and other components, such as, for example, one or more pharmaceutically acceptable carriers, adjuvants, and/or vehicles appropriate for the particular route of administration for which the composition is to be employed. In some embodiments, the carrier, adjuvant, and/or vehicle is suitable for injection (via a needle for example) for intravenous, intramuscular, intraperitoneal, transdermal, or subcutaneous administration, as well as a consumable, or spray for related oral and inhalant administrations.

Methods of the disclosure may include preparing a therapy formulation that includes the composition of the invention as well as other molecular species such as Tregs or Th1. Because the beneficial effects of those cells on attenuating an allergic response may arise from cell surface proteins or cellular/cytoplasmic contents of those cells (e.g., proteins, sugars, lipids, nucleic acids, salts, or combinations or complexes thereof), the formulation may include fragments or remnants of broken or ruptured cells such as those. Other molecular species such as histamine or cytokines such as IL-4R, IL-4, IL-13, IL-33, IL-9, IL-10, or IL-5 or TGF-beta, may be included in the formulations of the invention. The formulation may also include molecular species such as antibodies that bind to, sequester, block, or neutralize Th2 cells or cytokines such as IL-4 & IL-13. Compositions of the invention may include opsonins specific to Th2 cells that mark those cells for destruction. Therapeutic formulations of the composition may be given systemically by intravenous (IV) injection or by intramuscular (IM) and subcutaneous (SC) injection modes.

Preferably the therapeutic composition is formulated at a suitably high stable concentration with parameters such as viscosity optimized for a delivery route. For example, viscosity may be tuned to match a particular syringe or autoinjector. The formulation may comprise an aqueous solution or suspension with suitable buffers and optionally any other excipients to mitigate undesirable protein instability. Example excipients include fillers, extenders, diluents, solvents, preservatives, absorption enhancers and sustained release matrices. Buffers and excipients that are FDA approved for formulation of antibodies are generally known by those of skill in the art.

Once the therapeutic composition has been formulated, it may be provided for delivery to a patient who is potentially susceptible to an allergic reaction. For example, the composition may be packaged, e.g., in a bottle, vial, syringe, autoinjector, reservoir for an autoinjector or other device, or IV bag. The composition may be stored, e.g., in a cooler or freezer, or carried to a clinical setting for delivery. The composition may be packaged, e.g., in dry ice, and shipped to a hospital or other clinical setting for administration to a subject by a clinical professional.

In some embodiments, the composition of the invention is delivered directly in a prolonged release formulation. The composition itself may be modified to include features that increase serum half-life.

In another aspect, the invention provides for a method of treating allergies comprising providing to a subject having an allergy or at risk of having an allergic reaction a composition comprising an antibody or antigen-binding fragment thereof, and a binding moiety linked to the antibody or antigen-binding fragment thereof, wherein the binding moiety binds to a macromolecule within the patient after delivery into the patient. Specifically, the composition comprises a binding moiety linked to an antibody or antigen-binding fragment thereof, wherein the binding moiety binds to a macromolecule after in vivo delivery. The binding moiety portion of the composition binds to an endogenous in vivo molecular structure present in the body thus becoming a steric inhibitor coupled to the antibody or antigen-binding fragment, preventing IgE-mediated degranulation of mast cells and/or basophils. The binding moiety may bind to any macromolecule with a long chain, branched, multimeric, or quaternary molecular structure with sufficient size to inhibit further antigen binding to, and crosslinking of, IgE receptor complexes on mast cells and basophils. For example, the steric inhibitor may be, but is not limited a carbohydrate, protein, lipid, nucleic acid, oligonucleotide, long-chain lipid, or fatty acid.

In embodiments, the binding moiety linked to the antibody, or antigen-binding fragment thereof, is a ligand. The ligand may be an albumin-binding ligand such as described in Zorzi, A., 2017, Acylated heptapeptide binds albumin with high affinity and application as tag furnishes long-acting peptides, Nature Communications 8:16092, incorporated by reference herein. The ligand further may be a human serum albumin (HAS) which, once delivered, binds to fatty acids such as described in Fasano M, 2005, The extraordinary ligand binding properties of human serum albumin, IUBMB Life 57(12):787-96, incorporated by reference herein. Further, the ligand may be a lectin and, once delivered, binds to one or more sugars, as described in Raposo, D., 2021, Human lectins, their carbohydrate affinities and where to find them, Biomolecules 11, 188, incorporated by reference herein.

The antibody, or antigen-binding fragment thereof, of the composition of the method may be a monoclonal antibody, a polyclonal antibody, or a synthetic antibody such as a recombinant antibody, nucleic acid aptamer, or non-immunoglobulin antibody. Methods of making and purifying antibodies are known in the art and were developed by 1980s as described Harlow and Lane, 1988, Antibodies: A Laboratory Manual, CSHP, incorporated by reference.

For example, a monoclonal antibody is an antibody made by cloning a unique white blood cell, wherein all antibodies derived this way trace back to a unique parent cell. Monoclonal antibodies can have monovalent affinity, binding only to the same epitope. Monoclonal antibodies may be isolated or purified using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, Cold Spring Harbor Laboratory, incorporated by reference. See also Olsson, 1984, Human monoclonal antibodies: Methods of production and some aspects of their application in oncology, Med. Oncol. & Tumor Pharmacother 1, 235, incorporated by reference. These B cells are typically sourced from animals, usually mice. After cell fusion, large numbers of clones are screened and selected on the basis of antigen specificity and immunoglobulin class.

Such hybridomas may be stored frozen and cultured as needed to produce the specific monoclonal antibody. As is described below, monoclonal antibodies may be deployed therapeutically in methods of the invention. Those immunoglobulins exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid. It should be noted that the methods of deriving nucleic acids, including a nucleic acid sequence, encoding the allergen-specific antibody, derived from sequences identified from isolated single B cells from a human subject who is allergic to the specific allergen are described in International PCT Application No. PCT/US2019/032951 (published as WO 2019/222679), the disclosure of which is incorporated by reference herein in its entirety. In particular, such methods include combining single cell RNA sequencing (scRNA-seq) with functional antibody assays to elucidate mechanisms underlying the regulation of IgE and to discover high affinity, cross-reactive allergen-specific antibodies.

Polyclonal antibodies are made using several different immune cells and have affinity for the same antigen but different epitopes. Polyclonal antibodies may be prepared and purified by methods known to one skilled in the art such as by injecting an antigen/adjuvant conjugate into an animal of choice to initiate an amplified immune response, extracting blood, and purifying for the antibody of interest. Many methodologies exist for polyclonal antibody production in laboratory animals and are known to a person skilled in the art, such as is described in Dunbar, 1990, Preparation of polyclonal antibodies. Methods Enzymol 182:663-70, incorporated by reference. See also Newcombe C, 2007, Antibody production: polyclonal-derived biotherapeutics. J Chromatogr B Analyt Technol Biomed Life Sci. March 15; 848(1):2-7, incorporated by reference.

Synthetic antibodies include recombinant antibodies, nucleic acid aptamers, and non-immunoglobulin protein scaffolds. Synthetic antibodies may be purchased commercially. Methods for making and purifying synthetic antibodies are known in the art and can be found, for example, in Takeuchi T, 2018, Beyond natural antibodies—a new generation of synthetic antibodies created by post-imprinting modification of molecularly imprinted polymer, s. Chem Commun (Camb) 54(49):6243-6251, incorporated by reference. For example, recombinant antibodies are monoclonal antibodies generated in vitro using synthetic genes. Recombinant monoclonal antibodies may be purchased, or prepared by recovering antibody genes from source cells, amplifying and cloning the genes into an appropriate high-yield expression vector, and introducing the vector into an expression host, such as bacteria, yeast, or mammalian cell lines generate adequate amounts of functional antibodies.

Non-immunoglobulin derived synthetic antibodies may be generated either from nucleic acids, as in the case of aptamers or from non-immunoglobulin protein scaffolds/peptide aptamers, into which hypervariable loops are inserted to form the antigen binding site. Constraining the hypervariable binding loop at both ends within the protein scaffold improves the binding affinity and specificity of the synthetic antibody to levels comparable to or exceeding that of a natural antibody. Common advantages of these molecules compared to use of the typical antibody structure include a smaller size, giving improved tissue penetration, rapid generation times of weeks compared to months for natural and recombinant antibodies and cheaper costs.

Because an allergic reaction involves mast cells or basophils presenting IgE-bound Fc receptors that bind to and are cross-linked by IgE, in embodiments, compositions may include an antibody fragment, such as an IgE fragment, that may bind an allergen but that does not bind and cross-link Fc receptors. Antibody fragments are proteins that form part of the antigen recognition site. Antibody fragments may be produced in genetically modified bacteriophages, bacteria, fungi, or plants, such as is described in Joosten, V., 2003, The production of antibody fragments and antibody fusion proteins by yeasts and filamentous fungi, Microb Cell Fact 2, 1, incorporated by reference.

In other embodiments, the antibody or antigen-binding fragment thereof, is an immunoglobulin molecule selected from the group consisting of IgG, IgA, IgM, and a fragment thereof. Immunoglobulin G (IgG) is the most common type of antibody found in blood circulation. As noted above, IgG antibodies are created and released by plasma B cells. Each IgG antibody has two paratopes, or antigen-binding sites. Immunoglobulin A (IgA) is an antibody that plays a crucial role in the immune function of mucous membranes. The amount of IgA produced in association with mucosal membranes is greater than all other types of antibody combined. Immunoglobulin M (IgM) is the largest antibody, and it is the first antibody to appear in the response to initial exposure to an antigen. The IgG, IgA, IgM antibodies or fragment thereof, may be produced by any of the methods described above.

In some embodiments, the IgG antibody is a monoclonal antibody that binds to Ara h 1, Ara h 2, Ara h 3, or Ara h 6. Peanut causes one of the most serious food allergies. Ara h 1, Ara h 2, Ara h 3, and Ara h 6 belong to the peanut seed storage protein classes conarachin, conglutin and arachin. Ara h 1, Ara h 2, and Ara h 3 are classified as the major peanut allergens which can be recognized by more than 50% of peanut-allergic patients. See Burks W, Sampson H A, 1998, Peanut Allergens, Allergy 53:725-730, incorporated by reference herein. See also Rabjohn P, et al., 1999, Molecular cloning and epitope analysis of the peanut allergen Ara h 3, J Clin Invest 103:535-542, incorporated by reference herein; see also Barre A, et al., 2005, Molecular modelling of the major peanut allergen Ara h 1 and other homotrimeric allergens of the cupin superfamily: a structural basis for their IgE-binding cross-reactivity, Biochimie 87:499-506, incorporated by reference herein. Ara h 3 is recognized by serum IgE from approximately 44-54% of different patient populations with a history of peanut sensitivity. See Ratnaparkhe, M. B., et. al., 2014, Comparative and evolutionary analysis of major peanut allergen gene families, Genome biology and evolution 6(9), 2468-2488, incorporated by reference herein.

The methods are designed to alleviate and prevent an allergic response associated with specific allergens. In embodiments the allergen targeted comprises a food allergen. For example, the food allergen may be, but is not limited to, a milk allergen, an egg allergen, a nut allergen, a fish allergen, a shellfish allergen, a soy allergen, a legume allergen, a seed allergen, or a wheat allergen. In other embodiments, the allergen is pet allergen. In still other embodiments, the allergen is an environmental allergen such as, but not limited to, a plant allergen, a fungal allergen, a dust mite allergen, a drug allergen, a cosmetic allergen, or a latex allergen.

The composition of the method may be formulated for intranasal, transdermal, oral, or intravenous delivery. Further, the composition may be encapsulated in a microparticle, such as a lipid nanoparticle. As described in Lengyel, 2019, Microparticles, Microspheres, and Microcapsules for advanced drug delivery, Sci. Pharm 87, 20, incorporated by reference herein, and known to the skilled artisan, microparticles, microspheres, and microcapsules are widely used constituents of multiparticulate drug delivery systems. Microparticles are generally in the 1-1000 μm size range, serve as multiunit drug delivery systems with well-defined physiological and pharmacokinetic benefits in order to improve the effectiveness, tolerability, and patient compliance.

Methods of the invention provide for the administration of a therapeutically effective amount of a pharmaceutical formulation to a subject for preventing or treating an allergic response in the subject by providing the subject a dose of the composition. The formulation generally includes a composition comprising the composition and other components, such as, for example, one or more pharmaceutically acceptable carriers, adjuvants, and/or vehicles appropriate for the particular route of administration for which the composition is to be employed. In some embodiments, the carrier, adjuvant, and/or vehicle is suitable for injection (via a needle for example) for intravenous, intramuscular, intraperitoneal, transdermal, or subcutaneous administration, as well as a consumable, or spray for related oral and inhalant administrations.

Methods of the disclosure may include preparing a therapy that includes compositions comprising an antibody, a fragment thereof, a cytokine, or a cell, or a fragment thereof, coupled with a steric inhibiter that inhibits an allergic response. The composition may then be prepared for therapeutic delivery. Therapeutic formulations of the composition may be given systemically by intravenous (IV) injection or by intramuscular (IM) and subcutaneous (SC) injection modes.

Preferably the therapeutic composition is formulated at a suitably high stable concentration with parameters such as viscosity optimized for a delivery route. For example, viscosity may be tuned to match a particular syringe or autoinjector. The formulation may comprise an aqueous solution or suspension with suitable buffers and optionally any other excipients to mitigate undesirable protein instability. Example excipients include fillers, extenders, diluents, solvents, preservatives, absorption enhancers and sustained release matrices. Buffers and excipients that are FDA approved for formulation of antibodies are generally known by those of skill in the art.

Once the therapeutic composition has been formulated, it may be provided for delivery to a patient who is potentially susceptible to an allergic reaction. For example, the composition may be packaged, e.g., in a bottle, vial, syringe, autoinjector, reservoir for an autoinjector or other device, or IV bag. The composition may be stored, e.g., in a cooler or freezer, or carried to a clinical setting for delivery. The composition may be packaged, e.g., in dry ice, and shipped to a hospital or other clinical setting for administration to a subject by a clinical professional.

In some embodiments, the composition of the invention is delivered directly in a prolonged release formulation. The composition itself may be modified to include features that increase serum half-life.

Throughout the present description it is understood that methods of the inventions may be used to respond to, study, or treat allergies to any allergen, including but not limited to the following allergens; Ambrosia artemisiifolia (short ragweed) antigen E (Amb a 1); Ambrosia artemisiifolia (short ragweed) antigen K (Amb a 2); Ambrosia artemisiifolia (short ragweed) Ra3 antigen (Amb a 3); Ambrosia artemisiifolia (short ragweed) Ra5 antigen (Amb a 5); Ambrosia artemisiifolia (short ragweed) Ra6 antigen (Amb a 6); Ambrosia artemisiifolia (short ragweed) Ra7 antigen (Amb a 7); Ambrosia trifida (giant Ragweed) Ra5G antigen (Amb t 5); Artemisia vulgaris (mugwort) antigen (Art v 1); Artemisia vulgaris (mugwort) antigen (Art v 2); Helianthus annuus (sunflower) antigen (Hel a 1); Helianthus annuus (sunflower) profilin (Hel a 2); Mercurialis annua (annual Mercury) profilin (Mer a 1); Cynodon dactylon (Bermuda grass) antigen (Cyn d 1); Cynodon dactylon (Bermuda grass) antigen (Cyn d 7); Cynodon dactylon (Bermuda grass) profilin (Cyn d 12); Dactylis glomerata (orchard grass) AgDg1 antigen (Dac g 1); Dactylis glomerata (orchard grass) antigen (Dac g 2); Dactylis glomerata (orchard grass) antigen (Dac g 3); Dactylis glomerata (orchard grass) antigen (Dac g 5); Holcus lanatus (velvet Grass) antigen (Hol l 1); Lolium perenne (rye grass) group I antigen (Lol p 1); Lolium perenne (rye grass) group II antigen (Lol p 2); Lolium perenne (rye grass) group III antigen (Lol p 3); Lolium perenne (rye grass) group IX antigen (Lol p 5); Lolium perenne (rye grass) antigen (Lol p Ib); Lolium perenne (rye grass) trypsin (Lol p 11); Phalaris aquatica (canary grass) antigen (Pha a 1); Phleum pratense (timothy grass) antigen (Phl p 1); Phleum pratense (timothy grass) antigen (Phl p 2); Phleum pratense (timothy grass) antigen (Phl p 4); Phleum pratense (timothy grass) antigen Ag 25 (Phl p 5); Phleum pratense (timothy grass) antigen (Phl p 6); Phleum pratense (timothy grass) profilin (Phl p 12); Phleum pratense (timothy grass) polygalacturonase (Phl p 13); Poa pratensis (Kentucky blue grass) group I antigen (Poa p 1); Poa pratensis (Kentucky blue grass) antigen (Poa p 5); Sorghum halepense (Johnson grass) antigen (Sor h 1); Alnus glutinosa (alder) antigen (Aln g 1); Betula verrucosa (birch) antigen (Bet v 1); Betula verrucosa (birch) profilin (Bet v 2); Betula verrucose (birch) antigen (Bet v 3); Betula verrucosa (birch) antigen (Bet v 4); Betula verrucosa (birch) isoflavone reductase homologue (Bet v 5); Betula verrucosa (birch) cyclophilin (Bet v 7); Carpinus betulus (hornbeam) antigen (Car b 1); Castanea sativa (chestnut) Bet v 1 homologue (Cas s 1); Castanea sativa (chestnut) chitinase (Cas s 5); Corylus avelana (hazel) antigen (Cor a 1); Quercus alba (white oak) antigen (Que a 1); Cryptomeria japonica (sugi) antigen (Cry j 1); Cryptomeria japonica (sugi) antigen (Cry j 2); Juniperus ashei (mountain cedar) antigen (Jun a 1); Juniperus ashei (mountain cedar) antigen (Jun a 3); Juniperus oxycedrus (prickly juniper) calmodulin-like antigen (Jun o 2); Juniperus sabinoides (mountain cedar) antigen (Jun s 1); Juniperus virginiana (eastern red cedar) antigen (Jun v 1); Fraxinus excelsior (ash) antigen (Fra e 1); Ligustrum vulgare (privet) antigen (Lig v 1); Olea europea (olive) antigen (Ole e 1); Olea europea (olive) profilin (Ole e 2); Olea europea (olive) antigen (Ole e 3); Olea europea (olive) antigen (Ole e 4); Olea europea (olive) superoxide dismutase (Ole e 5); Olea europea (olive) antigen (Ole e 6); Syringa vulgaris (lilac) antigen (Syr v 1); Acarus siro (mite) fatty acid-binding protein (Aca s 13); Blomia tropicalis (mite) antigen (Blo t 5); Blomia tropicalis (mite) Bt11a antigen (Blo t 12); Blomia tropicalis (mite) Bt6 fatty acid-binding protein (Blo t); Dermatophagoides pteronyssinus (mite) antigen P1 (Der p 1); Dermatophagoides pteronyssinus (mite) antigen (Der p 2); Dermatophagoides pteronyssinus (mite) trypsin (Der p 3); Dermatophagoides pteronyssinus (mite) amylase (Der p 4); Dermatophagoides pteronyssinus (mite) antigen (Der p 5); Dermatophagoides pteronyssinus (mite) chymotrypsin (Der p 6); Dermatophagoides pteronyssinus (mite) antigen (Der p 7); Dermatophagoides pteronyssinus (mite) glutathione transferase (Der p 8); Dermatophagoides pteronyssinus (mite) collagenolytic serine prot (Der p 9); Dermatophagoides pteronyssinus (mite) tropomyosin (Der p 10); Dermatophagoides pteronyssinus (mite) apolipophorin like p (Der p 14); Dermatophagoides microceras (mite) antigen (Der m 1); Dermatophagoides farinae (mite) antigen (Der f 1); Dermatophagoides farinae (mite) antigen (Der f 2); Dermatophagoides farinae (mite) antigen (Der f 3); Dermatophagoides farinae (mite) tropomyosin (Der f 10); Dermatophagoides farinae (mite) paramyosin (Der f 11); Dermatophagoides farinae (mite) Mag 3, apolipophorin (Der f 14); Euroglyphus maynei (mite) apolipophorin (Eur m 14); Lepidoglyphus destructor (storage mite) antigen (Lep d 2.0101); Lepidoglyphus destructor (storage mite) antigen (Lep d 2.0102); Bos domesticus (cow) Ag3, lipocalin (Bos d 2); Bos domesticus (cow) alpha-lactalbumin (Bos d 4); Bos domesticus (cow) beta-lactalbumin (Bos d 5); Bos domesticus (cow) serum albumin (Bos d 6); Bos domesticus (cow) immunoglobulin (Bos d 7); Bos domesticus (cow) casein (Bos d 8); Canis familiaris (dog) antigen (Can f 1); Canis familiaris (dog) antigen (Can f 2); Canis familiaris (dog) albumin (Can f ?); Equus caballus (horse) lipocalin (Equ c 1); Equus caballus (horse) lipocalin (Equ c 2); Felis domesticus (cat) cat-1 antigen (Fel d 1); Mus musculus (mouse) MUP antigen (Mus m 1); Rattus norvegius (rat) antigen (Rat n 1); Alternaria alternata (fungus) antigen (Alt a 1); Alternaria alternata (fungus) antigen (Alt a 2); Alternaria alternata (fungus) heat shock protein (Alt a 3); Alternaria alternata (fungus) ribosomal protein (Alt a 6); Alternaria alternata (fungus) YCP4 protein (Alt a 7); Alternaria alternata (fungus) aldehyde dehydrogenase (Alt a 10); Alternaria alternata (fungus) enloase (Alt a 11); Alternaria alternata (fungus) acid ribosomal protein P1 (Alt a 12); Cladosporium herbarum (fungus) antigen (Cla h 1); Cladosporium herbarum (fungus) antigen (Cla h 2); Cladosporium herbarum (fungus) aldehyde dehydrogenase (Cla h 3); Cladosporium herbarum (fungus) ribosomal protein); Cladosporium herbarum (fungus) YCP4 protein (Cla h 5); Cladosporium herbarum (fungus) enolase (Cla h 6); Cladosporium herbarum (fungus) acid ribosomal protein P1 (Cla h 12); Aspergillus flavus (fungus) alkaline serine proteinase (Asp fl 13); Aspergillus Fumigatus (fungus) antigen (Asp f 1); Aspergillus Fumigatus (fungus) antigen (Asp f 2); Aspergillus Fumigatus (fungus) peroxisomal protein (Asp f 3); Aspergillus Fumigatus (fungus) antigen (Asp f 4); Aspergillus Fumigatus (fungus) metalloprotease (Asp f 5); Aspergillus Fumigatus (fungus) Mn superoxide dismutase (Asp f 6); Aspergillus Fumigatus (fungus) antigen (Asp f 7); Aspergillus Fumigatus (fungus) ribosomal protein P2 (Asp f 8); Aspergillus Fumigatus (fungus) antigen (Asp f 9); Aspergillus Fumigatus (fungus) aspartis protease (Asp f 10); Aspergillus Fumigatus (fungus) peptidyl-prolyl isomerase (Asp f 11); Aspergillus Fumigatus (fungus) heat shock protein P70 (Asp f 12); Aspergillus Fumigatus (fungus) alkaline serine proteinase (Asp f 13); Aspergillus Fumigatus (fungus) antigen (Asp f 15); Aspergillus Fumigatus (fungus) antigen (Asp f 16); Aspergillus Fumigatus (fungus) antigen (Asp f 17); Aspergillus Fumigatus (fungus) vacuolar serine (Asp f 18); Aspergillus niger (fungus) beta-xylosidase (Asp n 14); Aspergillus niger (fungus) antigen (Asp n 18); Aspergillus niger (fungus) vacuolar serine proteinase; Aspergillus oryzae (fungus) TAKA-amylase A (Asp o 2); Aspergillus oryzae (fungus) alkaline serine proteinase (Asp o 13); Penicillium brevicompactum (fungus) alkaline serine proteinase (Pen b 13); Penicillium citrinum (fungus) heat shock protein P70 (Pen c 1); Penicillium citrinum (fungus) peroxisomal membrane protein (Pen c 3); Penicillium citrinum (fungus) alkaline serine proteinase (Pen c 13); Penicillium notatum (fungus) N-acetyl glucosaminidase (Pen n 1); Penicillium notatum (fungus) alkaline serine proteinase (Pen n 13); Penicillium notatum (fungus) vacuolar serine proteinase (Pen n 18); Penicillium oxalicum (fungus) vacuolar serine proteinase (Pen o 18); Trichophyton rubrum (fungus) antigen (Tri r 2); Trichophyton rubrum (fungus) serine protease (Tri r 4); Trichophyton tonsurans (fungus) antigen (Tri t 1); Trichophyton tonsurans (fungus) serine protease (Tri t 4); Candida albicans (fungus) antigen (Cand a 1); Candida boidinii (fungus) antigen (Cand b 2); Malassezia furfur (fungus) antigen (Mal f 1); Malassezia furfur (fungus) MF1 peroxisomal membrane protein (Mal f 2); Malassezia furfur (fungus) MF2 peroxisomal membrane protein (Mal f 3); Malassezia furfur (fungus) antigen (Mal f 4); Malassezia furfur (fungus) antigen (Mal f 5); Malassezia furfur (fungus) cyclophilin homologue (Mal f 6); Psilocybe cubensis (fungus) antigen (Psi c 1); Psilocybe cubensis (fungus) cyclophilin (Psi c 2); Coprinus comatus (shaggy cap) antigen (Cop c 1); Coprinus comatus (shaggy cap) antigen (Cop c 2); Coprinus comatus (shaggy cap) antigen (Cop c 3); Coprinus comatus (shaggy cap) antigen (Cop c 5); Coprinus comatus (shaggy cap) antigen (Cop c 7); Aedes aegyptii (mosquito) apyrase (Aed a 1); Aedes aegyptii (mosquito) antigen (Aed a 2); Apis mellifera (honey bee) phospholipase A2 (Api m 1); Apis mellifera (honey bee) hyaluronidase (Api m 2); Apis mellifera (honey bee) melittin (Api m 4); Apis mellifera (honey bee) antigen (Api m 6); Bombus pennsylvanicus (bumble bee) phospholipase (Bom p 1); Bombus pennsylvanicus (bumble bee) protease (Bom p 4); Blattella germanica (German cockroach) Bd90k (Bla g 1); Blattella germanica (German cockroach) aspartic protease (Bla g 2); Blattella germanica (German cockroach) calycin (Bla g 4); Blattella germanica (German cockroach) glutathione transferase (Bla g 5); Blattella germanica (German cockroach) troponin C (Bla g 6); Periplaneta americana (American cockroach) Cr-PII (Per a 1); Periplaneta americana (American cockroach) Cr-PI (Per a 3); Periplaneta americana (American cockroach) tropomyosin (Per a 7); Chironomus thummi (midge) hemoglobin (Chi t 1-9); Chironomus thummi (midge) component III (Chi t 1.01); Chironomus thummi (midge) component IV (Chi t 1.02); Chironomus thummi (midge) component I (Chi t 2.0101); Chironomus thummi (midge) component IA (Chi t 2.0102); Chironomus thummi (midge) component II-beta (Chi t 3); Chironomus thummi (midge) component IIIA (Chi t 4); Chironomus thummi (midge) component VI (Chi t 5); Chironomus thummi (midge) component VIIA (Chi t 6.01); Chironomus thummi (midge) component IX (Chi t 6.02); Chironomus thummi (midge) component VIIB (Chi t 7); Chironomus thummi (midge) component VIII (Chi t 8); Chironomus thummi (midge) component X (Chi t 9); Dolichovespula maculata (white face hornet) phospholipase (Dol m 1); Dolichovespula maculata (white face hornet) hyaluronidase (Dol m 2); Dolichovespula maculata (white face hornet) antigen 5 (Dol m 5); Dolichovespula arenaria (yellow hornet) antigen 5 (Dol a 5); Polistes annularies (wasp) phospholipase A1 (Pol a 1); Polistes annularies (wasp) hyaluronidase (Pol a 2); Polistes annularies (wasp) antigen 5 (Pol a 5); Polistes dominulus (Mediterranean paper wasp) antigen (Pol d 1); Polistes dominulus (Mediterranean paper wasp) serine protease (Pol d 4); Polistes dominulus (Mediterranean paper wasp) antigen (Pol d 5); Polistes exclamans (wasp) phospholipase A1 (Pol e 1); Polistes exclamans (wasp) antigen 5 (Pol e 5); Polistes fuscatus (wasp) antigen 5 (Pol f 5); Polistes metricus (wasp) antigen 5 (Pol m 5); Vespa crabo (European hornet) phospholipase (Vesp c 1); Vespa crabo (European hornet) antigen 5 (Vesp c 5.0101); Vespa crabo (European hornet) antigen 5 (Vesp c 5.0102); Vespa mandarina (giant Asian hornet) antigen (Vesp m 1.01); Vespa mandarina (giant Asian hornet) antigen (Vesp m 1.02); Vespa mandarina (giant Asian hornet) antigen (Vesp m 5); Vespula flavopilosa (yellowjacket) antigen 5 (Ves f 5); Vespula germanica (yellowjacket) antigen 5 (Ves g 5); Vespula maculifrons (yellowjacket) phospholipase A1 (Ves m 1); Vespula maculifrons (yellowjacket) hyaluronidase (Ves m 2); Vespula maculifrons (yellowjacket) antigen 5 (Ves m 5); Vespula pennsylvanica (yellowjacket) (antigen 5Ves p 5); Vespula squamosa (yellowjacket) antigen 5 (Ves s 5); Vespula vidua (wasp) antigen (Ves vi 5); Vespula vulgaris (yellowjacket) phospholipase A1 (Ves v 1); Vespula vulgaris (yellowjacket) hyaluronidase (Ves v 2); Vespula vulgaris (yellowjacket) antigen 5 (Ves v 5); Myrmecia pilosula (Australian jumper ant) antigen (Myr p 1); Myrmecia pilosula (Australian jumper ant) antigen (Myr p 2); Solenopsis geminata (tropical fire ant) antigen (Sol g 2); Solenopsis geminata (tropical fire ant) antigen (Sol g 4); Solenopsis invicta (fire ant) antigen (Sol i 2); Solenopsis invicta (fire ant) antigen (Sol i 3); Solenopsis invicta (fire ant) antigen (Sol i 4); Solenopsis saevissima (Brazilian fire ant) antigen (Sol s 2); Gadus callarias (cod) allergen M (Gad c 1); Salmo salar (Atlantic salmon) parvalbumin (Sal s 1); Gallus domesticus (chicken) ovomucoid (Gal d 1); Gallus domesticus (chicken) ovalbumin (Gal d 2); Gallus domesticus (chicken) conalbumin; A22 (Gal d 3); Gallus domesticus (chicken) lysozyme (Gal d 4); Gallus domesticus (chicken) serum albumin (Gal d 5); Metapenaeus ensis (shrimp) tropomyosin (Met e 1); Penaeus aztecus (shrimp) tropomyosin (Pen a 1); Penaeus indicus (shrimp) tropomyosin (Pen i 1); Todarodes pacificus (squid) tropomyosin (Tod p 1); Haliotis Midae (abalone) antigen (Hal m 1); Apium graveolens (celery) Bet v 1 homologue (Api g 1); Apium graveolens (celery) profilin (Api g 4); Apium graveolens (celery) antigen (Api g 5); Brassica juncea (oriental mustard) 2S albumin (Bra j 1); Brassica rapa (turnip) prohevein-like protein (Bar r 2); Hordeum vulgare (barley) BMAI-1 (Hor v 1); Zea mays (maize, corn) lipid transfer protein (Zea m 14); Corylus avellana (hazelnut) Bet v 1 homologue (Cor a 1.0401); Malus domestica (apple) Bet v 1 homologue (Mal d 1); Malus domestica (apple) lipid transfer protein (Mal d 3); Pyrus communis (pear) Bet v 1 homologue (Pyr c 1); Pyrus communis (pear) profilin (Pyr c 4); Pyrus communis (pear) isoflavone reductase homologue (Pyr c 5); Oryza sativa (rice) antigen (Ory s 1); Persea americana (avocado) endochitinase (Pers a 1); Prunus armeniaca (apricot) Bet v 1 homologue (Pru ar 1); Prunus armeniaca (apricot) lipid transfer protein (Pru ar 3); Prunus avium (sweet cherry) Bet v 1 homologue (Pru av 1); Prunus avium (sweet cherry) thaumatin homologue (Pru av 2); Prunus avium (sweet cherry) profilin (Pru av 4); Prunus persica (peach) lipid transfer protein (Pru p 3); Sinapis alba (yellow mustard) 2S albumin (Sin a 1); Glycine max (soybean) HPS (Gly m 1.0101); Glycine max (soybean) HPS (Gly m 1.0102); Glycine max (soybean) antigen (Gly m 2); Glycine max (soybean) profilin (Gly m 3); Arachis hypogaea (peanut) vicilin (Ar a h 1); Arachis hypogaea (peanut) (conglutin Ar a h 2); Arachis hypogaea (peanut) glycinin (Ar a h 3); Arachis hypogaea (peanut) glycinin (Ar a h 4); Arachis hypogaea (peanut) (profilin Ar a h 5); Arachis hypogaea (peanut) conglutin homologue (Ar a h 6); Arachis hypogaea (peanut) conglutin homologue (Ar a h 7); Actinidia chinensis (kiwi) cysteine protease (Act c 1); Solanum tuberosum (potato) patatin (Sol t 1); Bertholletia excelsa (Brazil nut) 2S albumin (Ber e 1); Juglans regia (English walnut) 2S albumin (Jug r 1); Juglans regia (English walnut) vicilin (Jug r 2); Ricinus communis (castor bean) 2S albumin (Ric c 1); Anisakis simplex (nematode) antigen (Ani s 1); Anisakis simplex (nematode) paramyosin (Ani s 2); Ascaris suum (worm) antigen (Asc s 1); Aedes aegyptii (mosquito) apyrase (Aed a 1); Aedes aegyptii (mosquito) antigen (Aed a 2); Hevea brasiliensis (rubber) elongation factor (Hev b 1); Hevea brasiliensis (rubber) 1,3-glucanase (Hev b 2); Hevea brasiliensis (rubber) antigen (Hev b 3); Hevea brasiliensis (rubber) component of microhelix protein complex (Hev b 4); Hevea brasiliensis (rubber) antigen (Hev b 5); Hevea brasiliensis (rubber) hevein precursor (Hev b 6.01); Hevea brasiliensis (rubber) hevein (Hev b 6.02); Hevea brasiliensis (rubber) C-terminal fragment antigen (Hev b 6.03); Hevea brasiliensis (rubber) patatin homologue (Hev b 7); Hevea brasiliensis (rubber) profilin (Hev b 8); Hevea brasiliensis (rubber) enolase (Hev b 9); Hevea brasiliensis (rubber) Mn-superoxide dismut (Hev b 10); and Ctenocephalides felis (cat flea) antigen (Cte f 1). In addition, an allergen may be a component of a vaccine, such as preservatives (e.g., thimersosal, monosodium glutamate), adjuvants (e.g., aluminum, lipids, nucleic acid, polyethylene lycol), stabilizers (e.g., gelatins), residual cell culture materials (e.g., proteins, nucleic acids, yeast), residual inactivating ingredients (e.g., formaldehyde), and antibiotics. Preferred targets include food allergens such as from nuts, fish, milk, etc., as well as venoms, pollens, dander, latex, fungi, medicines (including antibiotics) and in particular peanut, milk, shellfish, tree nuts, egg, fin fish, wheat, soy, and sesame.

In addition, an allergen may be a component of a vaccine, such as preservatives (e.g., thimersosal, monosodium glutamate), adjuvants (e.g., aluminum, lipids, nucleic acid, polyethylene glycol), stabilizers (e.g., gelatins), residual cell culture materials (e.g., proteins, nucleic acids, yeast), residual inactivating ingredients (e.g., formaldehyde), and antibiotics. Preferred targets include food allergens such as from nuts, fish, milk, etc., as well as venoms, pollens, dander, latex, fungi, medicines (including antibiotics) and in particular peanut, milk, shellfish, tree nuts, egg, fin fish, wheat, soy, and sesame.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A composition for treating allergies, the composition comprising:
an antibody or antigen-binding fragment thereof that specifically binds an epitope on an allergen; and
a steric inhibitor coupled to said antibody or antigen-binding fragment that prevents IgE-mediated cross-linking and degranulation of mast cells and/or basophils.

2. The composition of claim 1, wherein the antibody or antigen-binding fragment thereof is an immunoglobulin molecule selected from the group consisting of IgG, IgA, IgM, and a fragment thereof.

3. The composition of claim 1, wherein the antibody is a monoclonal IgG antibody that binds to Ara h 1, Ara h 2, Ara h 3, or Ara h 6.

4. The composition of claim 1, wherein the antibody or antigen-binding fragment thereof is a recombinant antibody.

5. The composition of claim 1, wherein the steric inhibitor comprises polyethylene glycol.

6. The composition of claim 1, wherein the steric inhibitor comprises albumin.

7. The composition of claim 1, wherein the steric inhibitor is coupled to the antibody or antigen-binding fragment via a linker.

8. The composition of claim 1, wherein the steric inhibitor is a macromolecule.

9. The composition of claim 1, wherein the allergen comprises a food allergen.

10. The composition of claim 1, wherein the allergen comprises an animal allergen.

11. The composition of claim 1, wherein the allergen comprises an environmental allergen.

12. The composition of claim 1, wherein the composition is formulated for intranasal, transdermal, oral or intravenous delivery.

13. The composition of claim 1, wherein the composition is encapsulated in a microparticle.

14. The composition of claim 13, wherein the microparticle is a lipid nanoparticle.

15. A composition for treating allergies, the composition comprising:
an antibody or antigen-binding fragment thereof that specifically binds an epitope on an allergen; and
a binding moiety linked to the antibody or antigen-binding fragment thereof, wherein when the composition is delivered into a body, the binding moiety binds to a molecular structure present in the body thus becoming a steric inhibitor coupled to the antibody or antigen-binding fragment that prevents IgE-mediated cross-linking and degranulation of mast cells and/or basophils.

16. The composition of claim 15, wherein the binding moiety linked to the antibody or antigen-binding fragment is a ligand.

17. The composition of claim 16, wherein the ligand is an albumin-binding ligand.

18. The composition of claim 16, wherein the ligand is a human serum albumin.

19. The composition of claim 16, wherein the ligand is a lectin.

20. The composition of claim 15, wherein the molecular structure present in the body comprises a macromolecule.

21. The composition of claim 15, wherein the antibody or antigen-binding fragment thereof is an immunoglobulin molecule selected from the group consisting of IgG, IgA, IgM, and a fragment thereof.

22. The composition of claim 15, wherein the antibody is a monoclonal IgG antibody that binds to Ara h 1, Ara h 2, Ara h 3, or Ara h 6.

23. The composition of claim 15, wherein the antibody or antigen-binding fragment thereof is a recombinant antibody.

24. A method for treating allergies, the method comprising:
providing to a subject having an allergy or at risk of having an allergic reaction a composition comprising an antibody or antigen-binding fragment thereof that specifically binds an epitope on an allergen, and a steric inhibitor coupled to said antibody or antigen-binding fragment that prevents IgE-mediated degranulation of mast cells and/or basophils.

25. The method of claim 24, wherein the antibody or antigen-binding fragment thereof is an immunoglobulin molecule selected from the group consisting of IgG, IgA, IgM, and a fragment thereof.

26. The method of claim 24, wherein the antibody is a monoclonal IgG antibody that binds to Ara h 1, Ara h 2, Ara h 3, or Ara h 6.

27. The method of claim 24, wherein the antibody or antigen-binding fragment thereof is a recombinant antibody.

28. The method of claim 24, wherein the steric inhibitor comprises polyethylene glycol.

29. The method of claim 24, wherein the steric inhibitor comprises albumin.

30. The method of claim 24, wherein the steric inhibitor is a macromolecule.

31. The method of claim 24, wherein the allergen comprises a food allergen.

32. The method of claim 24, wherein the allergen comprises an animal allergen.

33. The method of claim 24, wherein the allergen comprises an environmental allergen.

34. The method of claim 24, wherein the composition is formulated for intranasal, transdermal, oral or intravenous delivery.

35. The method of claim 24, wherein the composition is encapsulated in a microparticle.

36. The method of claim 35, wherein the microparticle is a lipid nanoparticle.

37. The method of claim 24, further comprising an adjuvant, diluent or carrier.

38. The method of claim 24, further comprising administering a therapeutically effective amount of a formula of the composition to a subject.

39. A method for treating allergies, the method comprising:
providing to a subject having an allergy or at risk of having an allergic reaction a composition comprising an antibody or antigen-binding fragment thereof that specifically binds an epitope on an allergen, and a binding moiety linked to the antibody or antigen-binding fragment thereof, wherein when the composition is delivered into a body, the binding moiety binds to a molecular structure present in the body thus becoming a steric inhibitor coupled to the antibody or antigen-binding fragment that prevents IgE-mediated degranulation of mast cells and/or basophils.

40. The method of claim 39, wherein the binding moiety linked to the antibody or antigen-binding fragment is a ligand.

41. The method of claim 39, wherein the ligand is an albumin-binding ligand.

42. The method of claim 39, wherein the ligand is a human serum albumin.

43. The method of claim 39, wherein the ligand is a lectin.

44. The method of claim 39, wherein the molecular structure present in the body comprises a macromolecule.

45. The method of claim 39, wherein the antibody or antigen-binding fragment thereof is an immunoglobulin molecule selected from the group consisting of IgG, IgA, IgM, and a fragment thereof.

46. The method of claim 39, wherein the antibody is a monoclonal IgG antibody that binds to Ara h 1, Ara h 2, Ara h 3, or Ara h 6.

47. The method of claim 39, wherein the antibody or antigen-binding fragment thereof is a recombinant antibody.

48. The method of claim 39, wherein the allergen comprises a food allergen.

49. The method of claim 39, wherein the allergen comprises an animal allergen.

50. The method of claim 39, wherein the allergen comprises an environmental allergen.

51. The method of claim 39, wherein the composition is formulated for intranasal, transdermal, oral or intravenous delivery.

52. The method of claim 39, wherein the composition is encapsulated in a microparticle.

53. The method of claim 52, wherein the microparticle is a lipid nanoparticle.

54. The method of claim 39, further comprising an adjuvant, diluent or carrier.

55. The method of claim 39, further comprising administering a therapeutically effective amount of a formula of the composition to a subject.
